(19) United States
(12) Patent Application Publication
(10) Pub. No.: US 2026/0094682 A1
(43) Pub. Date: Apr. 2, 2026

(54) SYSTEM AND METHOD FOR AUTOMATIC GENERATION OF A RADIOLOGY REPORT

(71) Applicant: GE Precision Healthcare LLC, Waukesha, WI (US)

(72) Inventors: Nivedha SRINIVASAN, Chennai (IN); Mouleeswaran KUMAR, Bengaluru (IN)

(21) Appl. No.: 18/901,905

(22) Filed: Sep. 30, 2024

(51) Int. Cl. G16H 15/00; G06T 7/00; G10L 15/26; G16H 30/40

(52) U.S. Cl. CPC G16H 15/00; G06T 7/0012; G10L 15/26; G16H 30/40; G06T 2207/20084; G06T 2207/20112

(57) ABSTRACT

Various systems and methods are provided for automatically generating a radiology report. A medical image of a region of interest of a subject may be received. A structure of the region of interest of the subject may be segmented using a segmentation model and classified using a classification model. The medical imaging including the classified structure may be displayed via a user device of a radiologist. Speech data of the radiologist related to the classified structure may be received from the user device. Text data corresponding to the speech data may be generated using a natural language processing model. A radiology report corresponding to the medical image may be generated in a predetermined format using an artificial intelligence model. The radiology report including the medical image and an annotation including the generated text data in relation to the classified structure may be displayed via the user device of the radiologist.

### TECHNICAL FIELD

The present disclosure relates to a system and method for automatic generation of a radiology report. More specifically, the present disclosure relates to a system and method for automatic generation of a radiology report corresponding to a medical image in a predetermined format using an artificial intelligence (AI) model, a segmentation model, a classification model, and a natural language processing (NLP) model.

### BACKGROUND

A medical imaging device may perform medical imaging of a region of interest of a subject, and generate a medical image of the region of interest of the subject. A radiologist may review the medical image, and generate a radiology report. The radiology report may include, among other things, the medical image and an annotation of a structure of the region of interest. For instance, the annotation may identify whether the structure is normal, abnormal, potentially abnormal, benign, malignant, or the like.

A radiologist might be required to review medical images generated by various medical devices having different configurations and/or medical imaging modalities. For instance, a radiologist might review computed tomography (CT) images, magnetic resonance imaging (MRI) images, ultrasound images, X-ray system images, positron emission tomography (PET) images, or the like. Each of these medical imaging modalities might be associated with their own complexities and might require comprehensive knowledge on the part of the radiologist. Also, each of these medical imaging modalities might experience rapid development in underlying technology. Accordingly, a radiologist might be required to devote a significant amount of time to maintaining familiarity with respect to the protocols and nuances of the various medical imaging modalities. Further, a radiologist might be required to review a large number of medical images and generate a corresponding large number of radiology reports in a relatively tight time frame. Accordingly, the radiologist might experience fatigue, and/or might misinterpret a medical image or misdiagnose a subject. In this way, a radiology report may be inaccurate and/or incomplete in some instances. Further still, various radiologists might prepare radiology reports that do not adhere to a common standard. In these cases, the variations in the radiology reports might inhibit compatibility of the radiology reports across institutions, entities, or the like.

### SUMMARY

This summary introduces concepts that are described in more detail in the detailed description. It should not be used to identify essential features of the claimed subject matter, nor to limit the scope of the claimed subject matter.

In an aspect, a system may include a memory configured to store instructions; and one or more processors configured to execute the instructions to: receive a medical image of a region of interest of a subject; segment a structure of the region of interest of the subject using a segmentation model; classify the structure of the region of interest of the subject using a classification model; display the medical image including the classified structure via a user device of a radiologist; receive speech data, of the radiologist, related to the classified structure from the user device; generate text data corresponding to the speech data using a natural language processing model; generate a radiology report corresponding to the medical image in a predetermined format using an artificial intelligence model; and display the radiology report including the medical image and an annotation including the generated text data in relation to the classified structure via the user device of the radiologist.

In another aspect, a method may include receiving a medical image of a region of interest of a subject; segmenting a structure of the region of interest of the subject using a segmentation model; classifying the structure of the region of interest of the subject using a classification model; displaying the medical image including the classified structure via a user device of a radiologist; receiving speech data, of the radiologist, related to the classified structure from the user device; generating text data corresponding to the speech data using a natural language processing model; generating a radiology report corresponding to the medical image in a predetermined format using an artificial intelligence model; and displaying the radiology report including the medical image and an annotation including the generated text data in relation to the classified structure via the user device of the radiologist.

In yet another aspect, a non-transitory computer-readable medium may store instructions that, when executed by one or more processors, cause the one or more processors to: receive a medical image of a region of interest of a subject; segment a structure of the region of interest of the subject using a segmentation model; classify the structure of the region of interest of the subject using a classification model; display the medical image including the classified structure via a user device of a radiologist; receive speech data, of the radiologist, related to the classified structure from the user device; generate text data corresponding to the speech data using a natural language processing model; generate a radiology report corresponding to the medical image in a predetermined format using an artificial intelligence model; and display the radiology report including the medical image and an annotation including the generated text data in relation to the classified structure via the user device of the radiologist.

### BRIEF DESCRIPTION OF DRAWINGS

FIG. 1 is a diagram of an example system for automatic generation of a radiology report.

FIG. 2 is a diagram of example components of one or more devices of FIG. 1.

FIG. 3 is a diagram of example models of the platform of FIG. 1.

FIG. 4 is a flowchart of an example process for automatic generation of a radiology report.

FIGS. 5A-5G are diagrams of an example process for automatic generation of a radiology report.

FIG. 6 is a flowchart of an example process for training a segmentation model.

FIG. 7 is a flowchart of an example process for training a classification model.

FIG. 8 is a flowchart of an example process for training an NLP model.

FIG. 9 is a flowchart of an example process for training an AI model.

### DETAILED DESCRIPTION

As addressed above, a radiologist might be require to interpret medical images generated by various medical imaging devices according to various medical imaging modalities that each include their own complexities and intricacies. Further, as addressed above, a radiologist might be required to interpret a large number of medical images in a relatively tight time frame. Further still, as addressed above, a radiologist might prepare a radiology report that is customized or stylized to the radiologist's own preferences, which might inhibit compatibility of the radiology report across institutions, entities, radiologists, or the like. Accordingly, the radiology reports might be inaccurate, incomplete, non-standardized, or the like.

Some embodiments of the present disclosure provide for the automatic generation of a radiology report corresponding to a medical image in a predetermined format using an AI model, a classified structure that is segmented using a segmentation model and classified using a classification model, and generated text data that is generated using an NLP model and speech data. In this way, the generated radiology report may be more accurate, may be more comprehensive, may be generated more quickly and efficiently, and may be more standardized than as compared to other radiology reports.

In this way, some embodiments of the present disclosure provide an improvement in the technical field of medical imaging, and provide a technical improvement with respect to radiology reports. Further, some embodiments of the present disclosure enhance diagnostic accuracy by providing the ability to generate consistent and high-quality radiology reports, by providing the ability to detect missed anomalies, by reducing the likelihood of diagnostic errors, and by increasing patient diagnostic accuracy. Further still, some embodiments of the present disclosure increase efficiency of radiologists by providing the automatic generation of radiology reports, reduce workload, and reduce the amount of turnaround time for radiology report generation. Further still, some embodiments of the present disclosure may result in reduced operational costs and allow cost-savings through effective use of resources for healthcare providers via the improvements in efficiency and accuracy. Further still, some embodiments of the present disclosure provide the ability to highlight various anomalies via model training on similar cases and patterns from previous medical images, which exposes radiologists to a diverse range of cases and serves as an educational tool for radiologists.

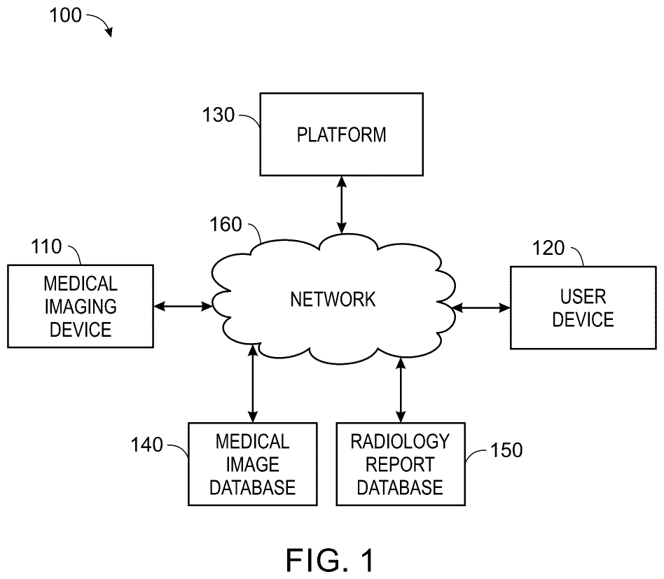

FIG. 1 is a diagram of an example system 100 for automatic generation of a radiology report. As shown in FIG. 1, the system 100 may include a medical imaging device 110, a user device 120, a platform 130, a medical imaging database 140, a radiology report database 150, and a network 160.

The medical imaging device 110 may be configured to acquire a medical image of a region of interest of a subject. For example, the medical imaging device 110 may be a CT device that is configured to acquire CT images, an MRI device that is configured to acquire MRI images, an ultrasound device that is configured to acquire ultrasound images, an X-ray device that is configured to acquire X-ray images, a PET device that is configured to acquire PET images, or the like. The subject may be a patient, an animal, a phantom, or the like. The region of interest may be any anatomical region of the subject. For example, the region of interest may be the brain, the heart, the liver, the pancreas, the kidneys, etc.

The user device 120 may be configured to display a medical image of a region of interest of a subject for viewing by a radiologist, receive speech data from the radiologist related to the medical image, display a radiology report for viewing by the radiologist, receive feedback from the radiologist on the radiology report, or the like. For example, the user device 120 may be a desktop computer, a laptop computer, a medical device, a tablet computer, a smartphone, or the like.

The platform 130 may be configured to receive a medical image of a region of interest of a subject, segment a structure of the region of interest of the subject using a segmentation model, classify the structure of the region of interest of the subject using a classification model, display the medical image including the classified structure via the user device 120 of a radiologist, receive speech data, of the radiologist, related to the classified structure from the user device 120, generate text data corresponding to the speech data using an NLP model, generate a radiology report corresponding to the medical image in a predetermined format using an AI model, display the radiology report including the medical image and an annotation including the generated text data in relation to the classified structure via the user device 120 of the radiologist, or the like. For example, the platform 130 may be a server, a computer, or the like.

The medical imaging database 140 may be configured to store medical images acquired by the medical imaging devices 110, store medical images segmented and/or classified by the platform 130, or the like. For example, the medical imaging database 140 may be a cloud database, a hierarchical database, a network database, a centralized database, a picture archiving and communication system (PACS), or the like.

The radiology report database 150 may be configured to store radiology reports generated by the platform 130, store radiology reports generated by radiologists, or the like. Further, the radiology report database 150 may store a template, or templates, for generating radiology reports. For example, the radiology report database 150 may be a cloud database, a hierarchical database, a network database, a centralized database, or the like.

The network 160 may permit communication between the medical imaging device 110, the user device 120, the platform 130, the medical imaging database 140, and/or the radiology report database 150. For example, the network 160 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional devices, fewer devices, different devices, or differently arranged devices than those shown in FIG. 1. Additionally, or alternatively, a set of devices (e.g., one or more devices) of the system 100 may be integrated into a single devices, and/or perform one or more functions described as being performed by another devices, or set of devices, of the system 100.

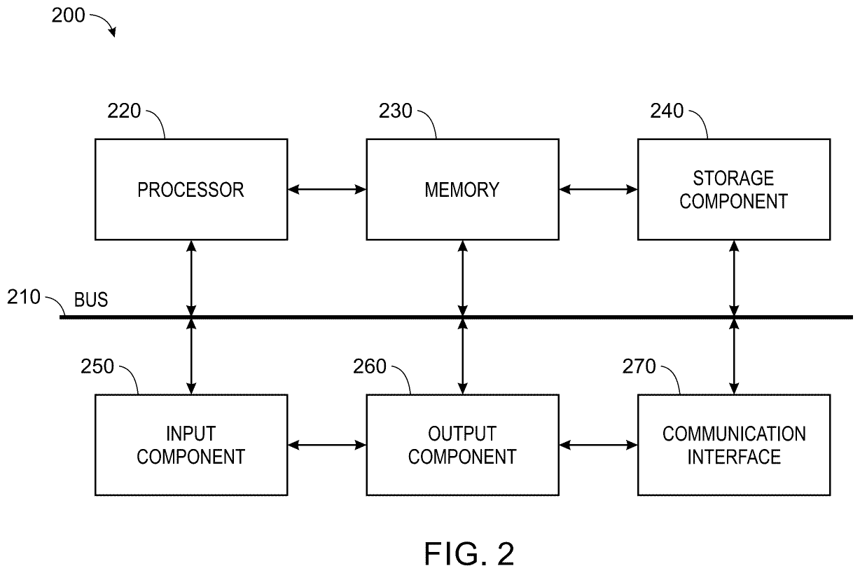

FIG. 2 is a diagram of example components of one or more devices 200 of FIG. 1. The device 200 may correspond to the medical imaging device 110, the user device 120, the platform 130, the medical imaging database 140, and/or the radiology report database 150. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 180 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 180 may include one or more processors capable of being programmed to perform a function. The processor 180 may include one or more processors 180 configured to perform the operations described herein. For example, a single processor 180 may be configured to perform all of the operations described herein. Alternatively, multiple processors 180, collectively, may be configured to perform all of the operations described herein, and each of the multiple processors 180 may be configured to perform a subset of the operations descried herein. For example, a first processor 180 may perform a first subset of the operations described herein, a second processor 180 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 180.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other systems, such as via a wired connection, a wireless connection, or a combination of wired and wireless connections. The communication interface 270 may permit the device 200 to receive information from another system and/or provide information to another system. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 180 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another system via the communication interface 270. When executed, the software instructions stored in the memory 230 and/or the storage component 240 may cause the processor 180 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components of the device 200 shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

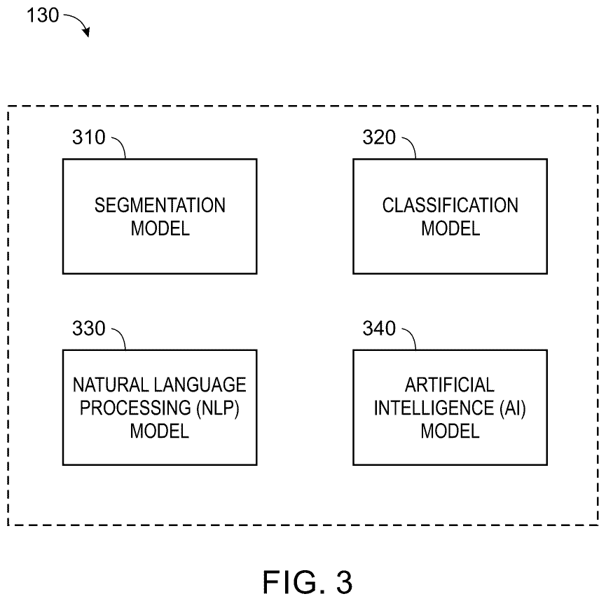

FIG. 3 is a diagram of example models of the platform of FIG. 1. As shown in FIG. 3, the platform 130 may include a segmentation model 310, a classification model 320, an NLP model 330, and an AI model 340.

The segmentation model 310 may be configured to receive a receive a medical image of a region of interest of a subject as an input, segment one or more structures in the region of interest, and output a segmented medical image in which the one or more structures are segmented. For example, the segmentation model 310 may be a convolutional neural network (CNN) model (e.g., a “U-Net model”), an edge-based segmentation model, a clustering-based segmentation model, a neural network-based segmentation model, a region-based segmentation model, or the like. According to a particular embodiment, the segmentation model 310 may be a U-Net model which is a CNN configured to image segmentation. The segmentation model 310 may include an encoder-decoder structure. The encoder may down-sample the medical image to capture context, and the decoder may up-sample the medical image to enable improved localization.

The classification model 320 may be configured to receive a medical image in which one or more structures are segmented as an input, classify the one or more segmented structures, and output a medical image in which the one or more segmented structures are classified. For example, the classification model 320 may be a residual neural network, a random forest model, a decision tree model, an artificial neural network (ANN), a Naïve Bayes model, or the like. According to a particular embodiment, the classification model 320 may be a residual neural network (which may be referred to as a “ResNet” model). The classification model 320 may use residual blocks to allow very deep networks which mitigates vanishing gradients. Further, the classification model 320 may use identity mappings in each block to preserve input information across layers.

The NLP model 330 may be configured to receive speech data of a radiologist as an input, generate text data corresponding to the speech data, and output the text data. For example, the NLP model 330 may be a generative pre-trained transformer model, a bidirectional encoder representations from transformers model, a large language model (LLM), an embeddings model, or the like.

The AI model 340 may be configured to receive a medical image in which one or more segmented structures are classified, receive text data relating to the one or segmented structures, and generate a radiology report corresponding to the medical image in a predetermined format. For example, the AI model 340 may be a decision tree (e.g., a classification tree, a regression tree, or the like), a linear regression model, a neural network (e.g., a deep neural network (DNN), a CNN, a recurrent neural network (RNN), an ANN, or the like), a logistic regression model, a support vector machine, or the like.

According to an embodiment, the segmentation model 310, the classification model 320, the NLP model 330, and/or the AI model 340 may be associated with a training phase, a deployment phase, and a monitoring phase. In the training phase, the platform 130 may receive and process training data to generate a trained model (which may any one or more of the foregoing models). The training data may be generated, received, or otherwise obtained from internal and/or external resources.

Generally, the trained model may include a set of variables (e.g., nodes, neurons, filters, or the like) that are tuned (e.g., weighted, biased, or the like) to different values via the application of the training data. According to an embodiment, the training process may employ supervised, unsupervised, semi-supervised, and/or reinforcement learning processes to train the model. According to an embodiment, a portion of the training data may be withheld during training and/or used to validate the trained model.

For supervised learning processes, the training data may include labels or scores that may facilitate the training process by providing a ground truth. For example, the labels or scores may indicate an output of the model. Training may proceed by feeding a training dataset including the training data into the model. The model may have variables set at initialized values (e.g., at random, based on Gaussian noise, based on pre-trained values, or the like). The model may generate an output based on the training dataset being input to the model. The output may be compared with the corresponding label or score (e.g., the ground truth) indicating the known output, which may then be back-propagated through the model to adjust the values of the variables. This process may be repeated for a plurality of samples at least until a determined loss or error is below a predefined threshold. According to an embodiment, some of the training data may be withheld and used to further validate or test the trained model.

For unsupervised learning processes, the training data may not include pre-assigned labels or scores to aid the learning process. Instead, unsupervised learning processes may include clustering, classification, or the like, to identify naturally occurring patterns in the training data. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data with pre-assigned labels or scores and training data without pre-assigned labels or scores may be used to train the model.

When reinforcement learning is employed, an agent (e.g., an algorithm) may be trained to make a decision from the training data through trial and error. For example, based on making a decision, the agent may then receive feedback (e.g., a positive reward if the prediction was above a predetermined threshold), adjust its next decision to maximize the reward, and repeat until a loss function is optimized.

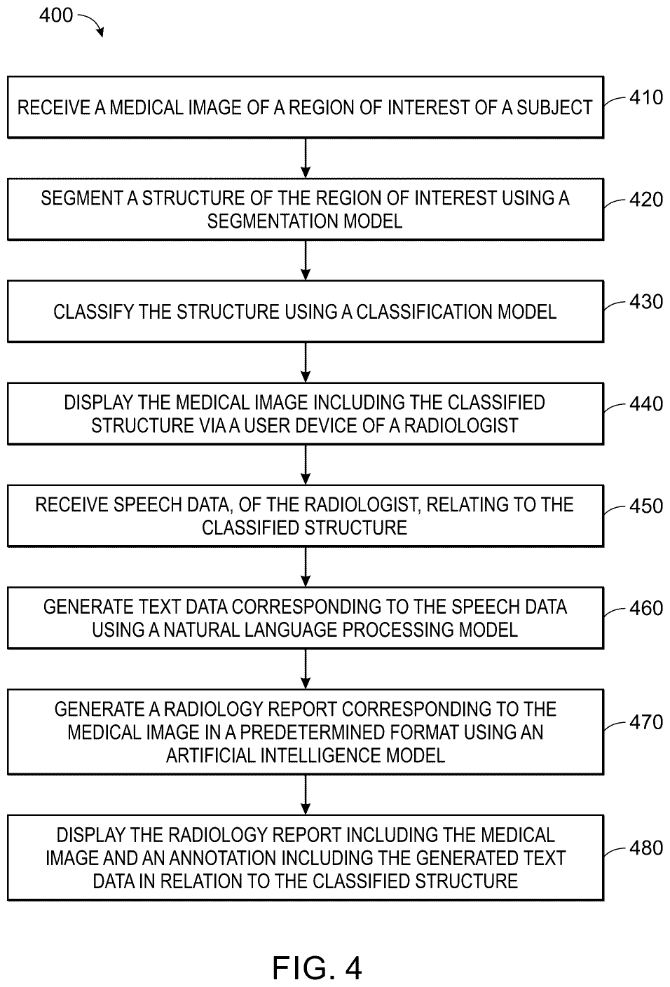

After being trained, the trained model may be stored and subsequently applied by the platform 130 during the deployment phase. For example, during the deployment phase, the trained model executed by the platform 130 may receive input data. During the deployment phase, the trained model may perform one or more operations as described in connection with FIG. 4.

After being deployed, the trained model may be monitored during the monitoring phase. For example, during the monitoring phase, the model may generate monitoring data 1016 that is used to monitor the trained model. The monitoring data may include data that identifies an output as determined by an operator. During the monitoring phase, monitoring data may be analyzed along with the predicted output data and input data to determine an accuracy of the trained model. According to an embodiment, based on the analysis, the process may return to the training phase, where values of one or more variables of the model may be adjusted to improve the accuracy of the model.

FIG. 4 is a flowchart of an example process 400 for automatic generation of a radiology report. According to an embodiment, the platform 130 may be configured to perform one or more operations of the process 400. Alternatively, one or more other devices of FIG. 1 may be configured to perform one or more operations of the process 400.

As shown in FIG. 4, the process 400 may include receiving a medical image of a region of interest of a subject (operation 410). For example, the platform 130 may receive a medical image of a region of interest of a subject from the medical imaging device 110, from the user device 120, from the medical imaging database 140, or the like. The medical image may be a CT image, an MRI image, an ultrasound image, an X-ray image, a PET image, or the like. The medical image may conform the digital imaging and communications in medicine (DICOM) standard. The subject may be a patient, an animal, a phantom, or the like. The region of interest may be any anatomical region of the subject. For example, the region of interest may be the brain, the heart, the liver, etc. According to an embodiment, the platform 130 may pre-process the medical image. For example, the platform 130 may adjust a resolution of the medical image to a resolution associated with the segmentation model 310 and/or the classification model 320. Further, the platform 130 may normalize pixel values between 0 and 1 in order to improve accuracy. Additionally, or alternatively, the platform 130 may enhance contrast, reduce noise, or the like.

As further show in FIG. 4, the process 400 may include segmenting a structure of the region of interest of the subject using a segmentation model (operation 420). For example, the platform 130 may segment the structure of the region of interest of the subject using the segmentation model 310. The platform 130 may input the medical image into the segmentation model 310, and receive a medical image including a segmentation result based on an output of the segmentation model 310. The segmentation result may include one or more segmented structures. According to an embodiment, the segmentation model 310 may generate a segmentation map that delineates the one or more structures of the region of interest. The structures may be tissues, vessels, tumors, lesions, anomalies, or the like. The segmentation map may include respective values for each pixel of the medical image. For example, the values may delineate a structure, and may indicate whether the structure is normal, abnormal, anomalous, or the like. According to an embodiment, the segmentation model 310 may post-process the medical image using thresholding, dilation, erosion, or the like, to remove noise and small artifacts.

As further show in FIG. 4, the process 400 may include classifying the structure of the region of interest of the subject using a classification model (operation 430). For example, the platform 130 may classify the structure of the region of interest of the subject using the classification model 320. The platform 130 may input the medical image that includes the segmentation result (e.g., the one or more segmented structures) into the classification model 320, and receive a medical image including a classification result based on an output of the segmentation model 310. The classification result may include one or more classified structures. The classification model 320 may extract high-level features of the one or more structures, and classify the one or more structures into one or more probability categories, such as benign, malignant, or the like. For instance, the classification result may include probability scores for each of the one or more structures. The probability scores may identify whether the structures belong to a particular classification category.

As further show in FIG. 4, the process 400 may include displaying the medical image including the classified structure via a user device of a radiologist (operation 440). For example, the platform 130 may display the medical image including the one or more classified structures via a user device 120 of a radiologist. The platform 130 may display the medical image including the segmentation result and the classification result. In this way, the radiologist may review the medical image including the one or more classified structures.

As further show in FIG. 4, the process 400 may include receiving speech data, of the radiologist, related to the classified structure from the user device (operation 450). For example, the platform 130 may receive speech data, of the radiologist, related to the classified structure from the user device 120. The radiologist may review the medical image including the one or more classified structures, and verbally comment on the one or more classified structures. The user device 120 may receive speech data of the radiologist based on the verbal comments via the input component 250 (e.g., microphone), and provide the speech data the platform 130.

As further show in FIG. 4, the process 400 may include generating text data corresponding to the speech data using a natural language processing model (operation 460). For example, the platform 130 may generate text data corresponding to the speech data using the NLP model 330. The platform 130 may input the speech data into the NLP model 330, and receive text data corresponding to the speech data based on an output of the NLP model 330. The NLP model 330 may be configured to extract key information such as information identifying a classified structure, information identifying a location of the classified structure in the region of interest, a measurement of the classified structure, or the like. The platform 130 may cross-reference the one or more segmented and classified structures with the extracted information to improve consistency and accuracy.

As further show in FIG. 4, the process 400 may include generating a radiology report corresponding to the medical image in a predetermined format using an artificial intelligence model (operation 470). For example, the platform 130 may generate a radiology report corresponding to the medical image in a predetermined format using the AI model 340. The platform 130 may input the medical image including the one or more classified structures and the text data into the AI model 340, and receive a radiology report corresponding to the medical image in a predetermined format. The predetermined format may correspond to a format that is associated with a template. For example, the template may identify a structure of the radiology report, and may identify respective information that is to be included in the radiology report.

According to an embodiment, the radiology report may include one or more sections. For example, the radiology report may include a type of exam section that identifies a type of imaging modality for the exam, a date of the exam, a time of the exam, or the like. Additionally, or alternatively, the radiology report may include a reason for exam section that identifies a reason for the exam, such as symptoms, previous medical history, or the like. Additionally, or alternatively, the radiology report may include a comparison section that includes information from a previous exam. Additionally, or alternatively, the radiology report may include a technique section that identifies how the exam was performed. Additionally, or alternatively, the radiology report may include a findings section that identifies what the radiologist sees in the medical image. For instance, the findings section may include a structure of the region of interest, and an associated description of the structure. As an example, the findings section may include a description of an area (e.g., “liver”), and an associated description of the structure (e.g., “normal”). Additionally, or alternatively, the radiology report may include an impression section that identifies a summary of the findings, potential causes, differential diagnoses, recommendations, or the like. Additionally, or alternatively, the radiology report may include the medical image. For example, the radiology report may include the medical image, the medical image including a segmentation result, the medical image including a classification result, or the like.

The template may identify the one or more sections of the radiology report. The AI model 340 may generate the radiology report using the template, such that the generated radiology report includes the respective information for each section. The AI model 340 may access medical records of the subject, previous medical images of the subject, previous radiology reports of the subject, or the like, to generate the radiology report. Additionally, or alternatively, the AI model 340 may access medical records of other subjects, previous radiology reports of other subjects, or the like, to generate the radiology report. In this way, the generated radiology report conforms to a predetermined format, and is more consistent with previously-generated radiology reports than as compared to manually prepared radiology reports.

The AI model 340 may generate the radiology report to include text data that conforms to the predetermined format. For example, the predetermined format may identify a sentence structure of text to be included in the radiology report. The sentence structure may be a standardized sentence structure. The AI model 340 may use the text data generated by the NLP model 330 and the sentence structure to generate the radiology report in the predetermined format. For instance, the text data may include a different sentence structure than the sentence structure of the predetermined format. In this case, the AI model 340 may generate the radiology report by changing the sentence structure of the text data to match the sentence structure of the predetermined format. In other words, the verbal comments of the radiologist might not conform to the sentence structure in some instances. In these cases, the AI model 340 may generate text data for the radiology report that conforms to the sentence structure. As an example, a radiologist may provide a verbal comment of “no abnormalities found in the liver.” Further, the AI model 340 may generate the radiology report to include the sentence of “the liver is normal.”

The radiology report may identify one or more structures of the region of interest, and may include respective annotations for the one or more structures. The annotations may identify a classification of the one or more structures. The AI model 340 may identify a structure based on a segmentation result and/or a classification result, and may identify text data that corresponds to the structure. The AI model 340 may generate an annotation for the structure using the text data. For example, as addressed above, the AI model 340 may generate an annotation for a liver that indicates that “the liver is normal.” In some cases, the AI model 340 might identify that no text data exists for a structure. For example, the radiologist might not comment on every single structure of the region of interest. In these cases, the AI model 340 may generate an annotation for the structure based on predetermined information. For example, the AI model 340 may generate an annotation that indicates “normal,” “no abnormalities,” or the like.

According to an embodiment, the AI model 340 may determine a diagnosis based on a segmentation result and/or a classification result, and generate the radiology report to include the diagnosis. Additionally, or alternatively, the AI model 340 may compare the radiology report with a previous radiology report, and generate a comparison result to be included in the radiology report. Additionally, or alternatively, the AI model 340 may compare a medical image with a previous medical image, and generate a comparison result to be included in the radiology report.

As further show in FIG. 4, the process 400 may include displaying the radiology report including the medical image and an annotation including the generated text data in relation to the classified structure via the user device of the radiologist (operation 480). For example, the platform 130 may display the radiology report including the medical image and an annotation including the generated text data in relation to the classified structure via the user device 120 of the radiologist. The radiologist may view the generated radiology report via the user device 120 (e.g., via a DICOM viewer, an AW server, a web-based interface, or the like), and may review, edit, or approve the radiology report.

According to an embodiment, the platform 130 may perform one or more actions based on generating the radiology report. For example, the platform 130 may transmit an alert to a user device 120 based on the radiology report. In this case, the alert may identify a diagnosis in the radiology report, an annotation from a radiologist, or the like. As another example, the platform 130 may automatically schedule an appointment for a follow-up, for medical imaging, for a medical procedure, or the like. In this case, the platform 130 may identify a medical practitioner to perform the follow-up, to perform the medical imaging, to perform the medical procedure, or the like, and schedule the appointment accordingly. As another example, the platform 130 may transmit the radiology report to a user device 120 associated with another medical personnel, to a user device 120 associated with the subject, or the like. As another example, the platform 130 may transmit the radiology report to the radiology report database 150, to the medical imaging database 120, or the like.

Although FIG. 4 depicts particular operations and a particular sequence of operations, it should be understood that other embodiments may include different operations or differently arranged operations than as shown in FIG. 4.

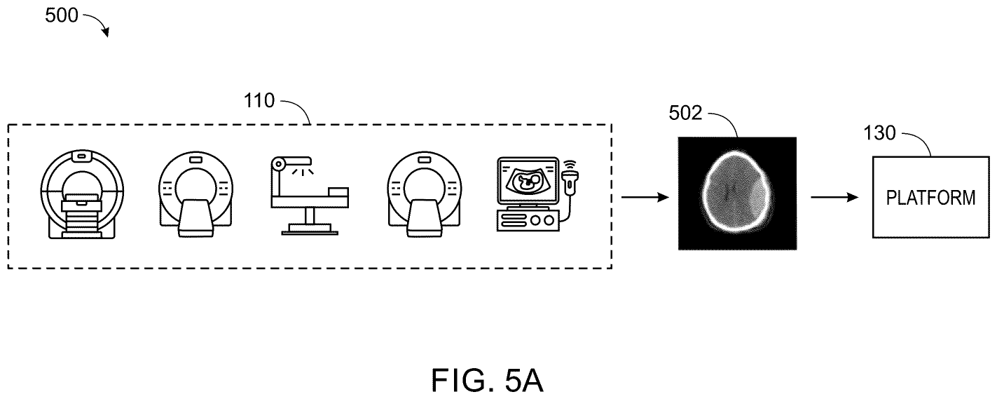

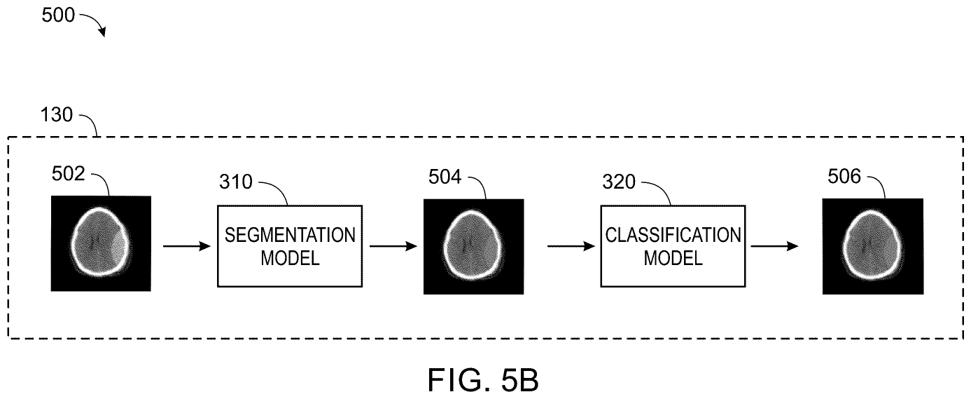

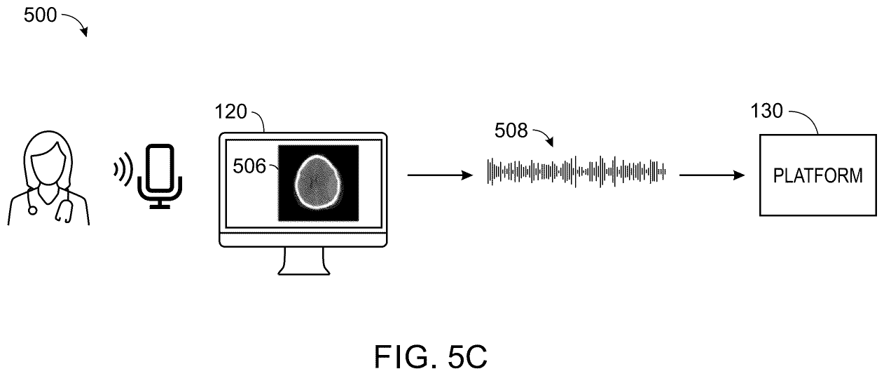

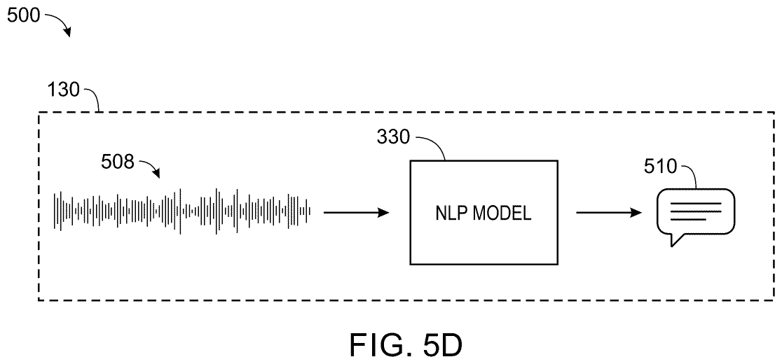

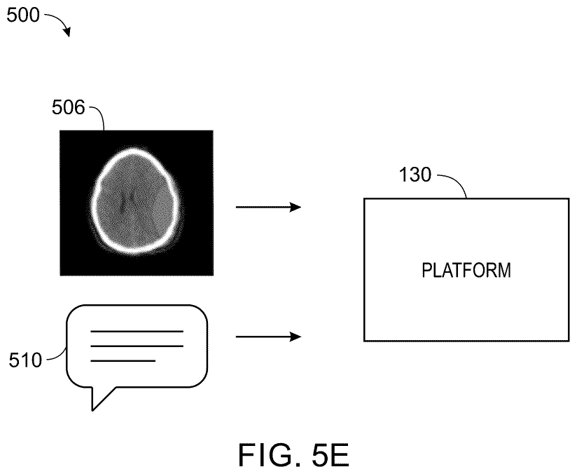

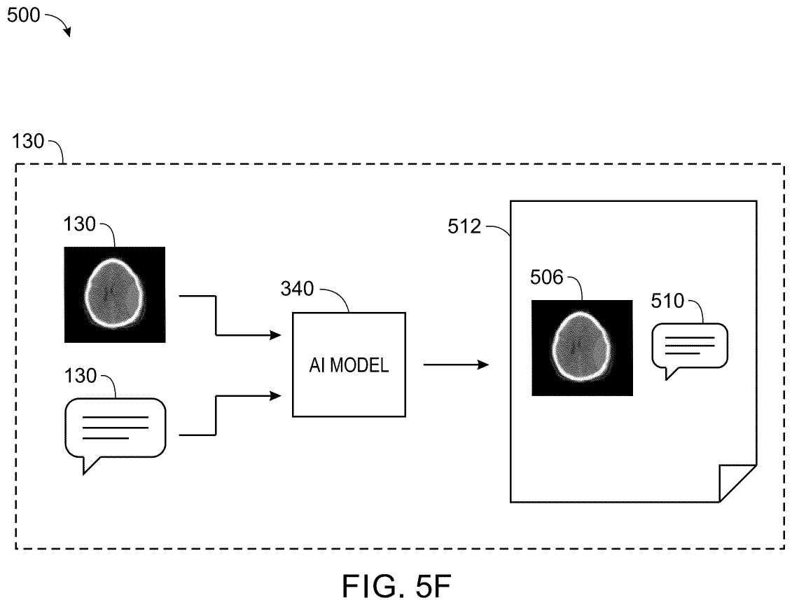

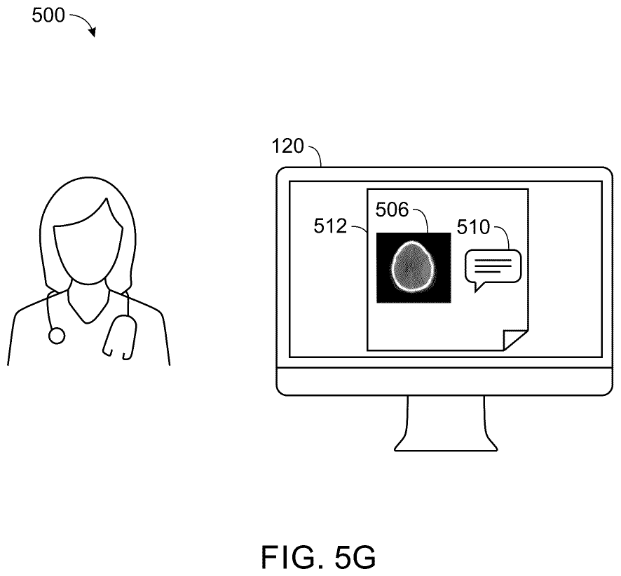

FIGS. 5A-5F are diagrams of an example process 500 for automatic generation of a radiology report. As shown in FIG. 5A, a medical imaging device 110 may acquire a medical image 502 of a region of interest of a subject, and provide the medical image 502 to the platform 130. As shown in FIG. 5B, the platform 130 may input the medical image 502 into the segmentation model 310, and receive a medical image 504 including a segmented structure based an output of the segmentation model 310. As further shown in FIG. 5B, the platform 130 may input the medical image 504 including the segmented structure into the classification model 320, and receive a medical image 506 including a classified structure based an output of the classification model 320. As shown in FIG. 5C, the platform 130 may display the medical image 506 including the classified structure via the user device 120 for display to a radiologist. As further shown in FIG. 5C, the user device 120 may receive speech data 508 based on verbal comments by the radiologist received via an input component 250 (e.g., microphone) of the user device 120. As further shown in FIG. 5C, the platform 130 may receive the speech data 508 from the user device 120. As shown in FIG. 5D, the platform 130 may input the speech data 508 into the NLP model 330, and receive text data 510 corresponding to the speech data based on an output of the NLP model 330. As shown in FIG. 5E, the platform 130 may receive the medical image 506 including the classified structure, and the text data 510 corresponding to the speech data of the radiologist relating to the classified structure. As shown in FIG. 5F, the platform 130 may input the medical image 506 including the classified structure and the text data 510 corresponding to the speech data of the radiologist into the AI model 340, and receive a radiology report 512 including the medical image 506 and an annotation 510 including the generated text data in relation to the classified structure based an output of the AI model 340. As shown in FIG. 5G, the platform 130 may display the radiology report 512 including the medical image 506 and the annotation 510 including the generated text data in relation to the classified structure via the user device 120 of the radiologist.

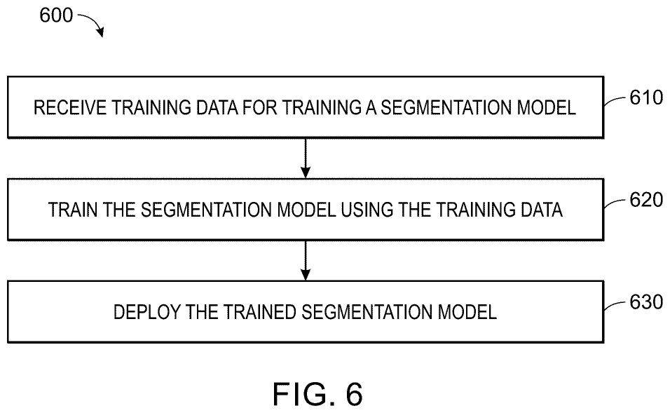

FIG. 6 is a flowchart of an example process for training a segmentation model. According to an embodiment, the platform 130 may be configured to perform one or more operations of the process 600. Alternatively, one or more other devices of FIG. 1 may be configured to perform one or more operations of the process 600.

As shown in FIG. 6, the process 600 may include receiving training data for training a segmentation model (operation 610). For example, the platform 130 may receive training data for training the segmentation model 310. The training data may include medical images (e.g., CT images, MRI images, X-ray images, ultrasound images, etc.) including annotations that describe one or more structures (e.g., tumors, lesions, tissues, etc.) in the medical images. For example, the annotations may include pixel-level labels for the one or more structures. The platform 130 may preprocess the training data by normalizing the pixel values for consistent input, resizing the medical images to a standard resolution for processing by the segmentation model 310, augmenting (e.g., rotating, scaling, flipping, etc.) the data to increase training variability, or the like.

As further shown in FIG. 6, the process 600 may include training the segmentation model using the training data (operation 620). For example, the platform 130 may train the segmentation model 310 using the training data. The segmentation model 310 may include an encoder-decoder structure. The encoder may capture context via downsampling, and may use convolutional layers with rectified linear unit (ReLU) activation and max pooling. The decoder may enable precise localization through upsampling using transpose convolutions concatenated with corresponding encoder features via skip connections. The platform 130 may split the medical images into a training dataset, a validation dataset, and a test dataset. The platform 130 may train the segmentation model 310 using GPUs, and by applying techniques such as early stopping, learning rate scheduling, or the like. The platform 130 may validate the performance of the segmentation model 310 using metrics, such as intersection over union (IoU), Dice coefficient, or the like, The platform 130 may optimize the segmentation model 310 by tuning hyperparameters (e.g., learning rate, batch size, number of epochs, or the like), and by using techniques such as dropout and batch normalization to prevent overfitting.

As further shown in FIG. 6, the process 600 may include deploying the trained segmentation model (operation 630). For example, the platform 130 may deploy the trained segmentation model 310. The platform 130 may deploy the trained segmentation model 310 by integrating the trained segmentation model 310 with the system 100, and provide application programming interfaces (APIs) to handle the input of medical images into the segmentation model 310, preprocessing of the medical images by the segmentation model 310, and output of segmented medical images by the segmentation model 310. The platform 130 may periodically update the segmentation model 310 with new training data. Further, the platform 130 may update the segmentation model 310 using feedback data. Further still, the platform 130 may use active learning to prioritize uncertain cases for manual annotation.

Although FIG. 6 depicts particular operations and a particular sequence of operations, it should be understood that other embodiments may include different operations or differently arranged operations than as shown in FIG. 6.

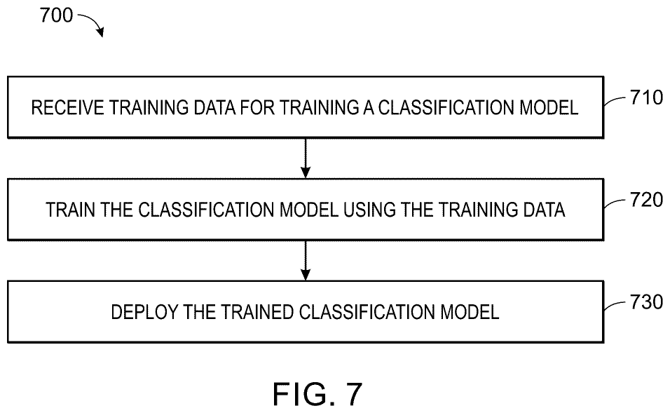

FIG. 7 is a flowchart of an example process for training a classification model. According to an embodiment, the platform 130 may be configured to perform one or more operations of the process 700. Alternatively, one or more other devices of FIG. 1 may be configured to perform one or more operations of the process 700.

As shown in FIG. 7, the process 700 may include receiving training data for training a classification model (operation 710). For example, the platform 130 may receive training data for training the classification model 320. The training data may include medical images including segmented structures. Each segmented structure may be labelled with a corresponding classification label (e.g., benign, malignant, healthy, or the like). The platform 130 may preprocess the training data by normalizing pixel values of the medical images, resizing segmented structures of the medical images to permit processing by the classification model 320, augmenting the training data to improve generalization of the classification model 320, or the like. The segmentation model 310 may utilize residual blocks to allow for very deep networks by mitigating the vanishing gradient problem. Each block may include identity mappings to preserve input information across layers. The platform 130 may split the medical images into a training dataset, a validation dataset, and a test dataset.

As further shown in FIG. 7, the process 700 may include training the classification model using the training data (operation 720). For example, the platform 130 may train the classification model using the training data. The platform 130 may use categorical cross-entropy loss for classification. The platform 130 may train the classification model using GPUs with early stopping, learning rate scheduling, or the like. The platform 130 may validate the performance of the classification model 320 using metrics, such as accuracy, precision, recall, F1 score, or the like. The platform 130 may optimize the classification model 320 by tuning hyperparameters, implanting regularization techniques (e.g., dropout, weight decay, or the like), or the like.

As further shown in FIG. 7, the process 700 may include deploying the trained segmentation model (operation 730). For example, the platform 130 may deploy the trained segmentation model 310. The platform 130 may deploy the trained segmentation model 310 by integrating the trained classification model 320 with the system 100. The platform 130 may provide APIs to handle the input of segmented medical images into the classification model 320, preprocessing of medical images by the classification model 320, and output of classified medical images by the classification model 320. The platform 130 may periodically update the classification model 320 with new annotated medical images and feedback data to improve accuracy. The platform 130 may implement active learning to improve the performance of the classification model 320 on challenging or uncertain cases.

Although FIG. 7 depicts particular operations and a particular sequence of operations, it should be understood that other embodiments may include different operations or differently arranged operations than as shown in FIG. 7.

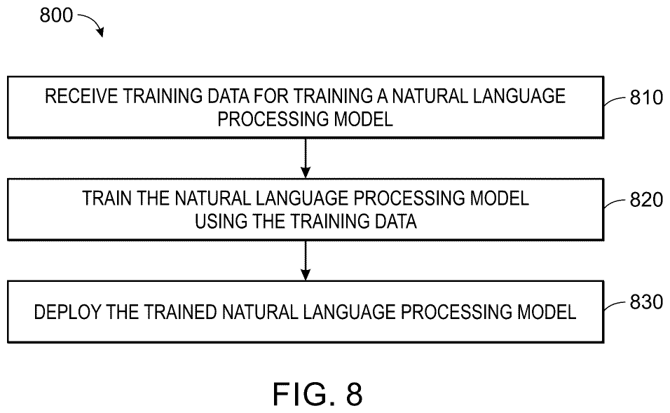

FIG. 8 is a flowchart of an example process for training an NLP model. According to an embodiment, the platform 130 may be configured to perform one or more operations of the process 800. Alternatively, one or more other devices of FIG. 1 may be configured to perform one or more operations of the process 800. As shown in FIG. 8, the process 800 may include receiving training data for training an NLP model (operation 810). For example, the platform 130 may receive training data for training the NLP model 330. The training data may include speech data and corresponding text data. As further shown in FIG. 8, the process 800 may include training the NLP model using the training data (operation 820). For example, the platform 130 may train the NLP model 330 using the training data. The platform 130 may train the NLP model 330 to receive speech data, and generate text data corresponding to the speech data. As further shown in FIG. 8, the process may include deploying the trained NLP model (operation 830). For example, the platform 130 may deploy the trained NLP model 330 by integrating the trained NLP model 330 with the system 100. Although FIG. 8 depicts particular operations and a particular sequence of operations, it should be understood that other embodiments may include different operations or differently arranged operations than as shown in FIG. 8.

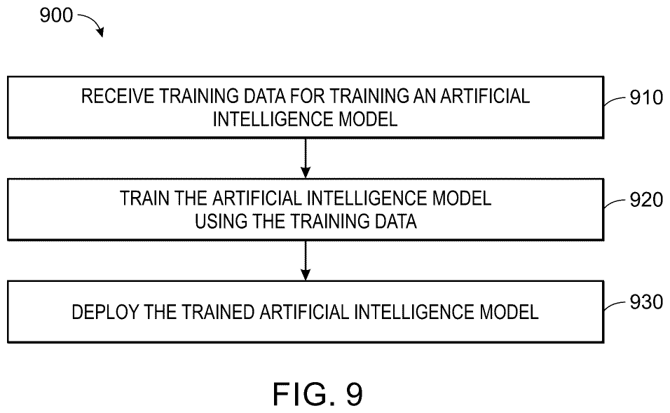

FIG. 9 is a flowchart of an example process for training an AI model. According to an embodiment, the platform 130 may be configured to perform one or more operations of the process 900. Alternatively, one or more other devices of FIG. 9 may be configured to perform one or more operations of the process 900.

As shown in FIG. 9, the process 900 may include receiving training data for training an AI model (operation 910). For example, the platform 130 may receive training data for training the AI model 340. The training data may include radiology reports including medical images and annotations. The medical images may be segmented and classified. The training data may include diverse cases to cover a large number of health conditions.

As further shown in FIG. 9, the process 900 may include training the AI model 340 using the training data (operation 920). For example, the platform 130 may train the AI model 340 using the training data. The platform 130 may train the AI model 340 using the training data to specialize in the generation of radiology reports. The platform 130 may use supervised learning with human-annotated examples to train the AI model 340 to generate radiology reports in a manner that conforms to a writing style of radiologists to and to conform to medical terminology. The platform 130 may use a high-performance computing cluster for training, and may implement gradient accumulation and mixed-precision training to handle large models. The platform 130 may be validated using a separate validation dataset. For example, the platform 130 may use metrics like BLEU score, ROUGE score, domain-specific evaluations by radiologists, or the like.

As further shown in FIG. 9, the process 900 may include deploying the trained AI model (operation 930). For example, the platform 130 may deploy he trained AI model 340. The platform 130 may deploy the AI model 340 by integrating the AI model 340 with the system 100. For example, the platform 130 may deploy the AI model 340 by integrating the AI model 340 with the segmentation model 310, the classification model 320, and/or the NLP model 330. The platform 130 may develop APIs to handle the upload and preprocessing of medical images, the segmentation of the medical images using the segmentation model 310, the classification of the medical images using the classification model 320, the generation of text data based on speech data of radiologists using the NLP model 330, and the generation of radiology reports using the AI model 340.

After integrating the AI model 340 with the segmentation model 310, the classification model 320, and/or the NLP model 330, the platform 130 may receive a medical image. The platform 130 may normalize the image, resize the image, or the like. The platform 130 may use the segmentation model 310 to generate a segmented medical image including a segmentation map. The platform 130 may apply post-processing techniques (e.g., thresholding, morphological operations, or the like). The platform 130 may use the classification model 320 to generate a classified medical image. For example, the classification model 320 may extract a segmented structure that was segmented by the segmentation model 310, and classify the segmented structure. The platform 130 may receive speech data of a radiologist, and generate text data corresponding to the speech data using the NLP model 330. The platform 130 may generate a radiology report using the AI model 340, the medical image, the segmented medical image, the classified medical image, and/or the text data. The platform 130 may display the radiology report via the user device 120 of the radiologist to permit the radiologist to review, edit, or approve the radiology report.

The platform 130 may receive feedback data related to the segmented medical mage, the classified medical image, the text data, and/or the radiology report. The platform 130 may implement a continuous learning pipeline in which the platform 130 may update the segmentation model 310, the classification model 320, the NLP model 330, and/or the AI model 340 based on new training data and/or feedback data. The platform 130 may use active learning to prioritize uncertain cases for manual review and/or annotation. The platform 130 may monitor model performance in real-time, and implement alert mechanisms for model drift or anomalies in outputs. Although FIG. 9 depicts particular operations and a particular sequence of operations, it should be understood that other embodiments may include different operations or differently arranged operations than as shown in FIG. 9.

Some embodiments of the present disclosure provide enhanced diagnostic accuracy. For example, the embodiments may use the segmentation model 310 and the classification model 320, and integrate these models with the AI model 340, which enhances diagnostic accuracy and patient outcomes in radiology. Further, some embodiments of the present disclosure provide reduced interpretation time and improved efficiency. For example, the embodiments may significantly reduce interpretation time for radiologists, which results in faster diagnosis, reduced patient waiting times, ensures timey initiation of treatments, and improved patient satisfaction and overall efficiency. Further still, some embodiments of the present disclosure provide automatic detection and segmentation of structures associated with anomalies in medical images. For example, by using the segmentation model 310 and the classification model 320, the embodiments may detect and highlight structures associated with anomalies, which reduces the like. Further still, some embodiments of the present disclosure provide contextual insights and enhanced radiology report generation. For instance, the integration of the NLP model 330 and the AI model 340 provides contextual insights derived from similar cases, which leads to informed decision-making, increases diagnostic confidence in complex and rare cases. Further still, some embodiments of the present disclosure provide information extraction from unstructured comments by radiologists. For example, the embodiments herein may process and extract relevant information from unstructured speech data from radiologists, which reduces workload. Further still, some embodiments herein provide continuous learning and adaptation. For instance, the embodiments incorporate feedback from radiologists to enhance accuracy, relevance, and usability through continuous feedback mechanisms, which improves ongoing improvement and adaptation to evolving medical standards. Further, the continuous learning can improve performance over time, which can significantly improve diagnostic accuracy.

According to an example use case, a radiologist may review an MRI image of a brain for suspected multiple sclerosis. The radiologist may interact with the user device 120 to upload the MRI image to the platform 130. The platform 130 may pre-process the MRI image, such as by enhancing contrast and reducing noise. The platform 130 may segment the MRI image of a brain into various structures (e.g., white matter, grey matter, cerebrospinal fluid, anomalous structures, etc.) using the segmentation model 310. The platform 130 classify the structures using the classification model 320. The platform 130 may identify potential demyelinating lesions based on the classification. The platform 130 may assign probability scores to the classified structures, which indicates probability of the structures being anomalous. The platform 130 may display the medical image including a segmentation result and a classification result via the user device 120, and receive speech data of the radiologist regarding the structures from the user device 120. The platform 130 may generate text data related to the speech data using the NLP model 330, and generate annotations for the text data that correspond to the medical image. The platform 130 may integrate the annotations including the text data and the medical image into a comprehensive radiology report. The radiology report may include auto-generated annotations for areas on which the radiologist did not comment, which identifies the areas as being normal or healthy. The radiology repot may include annotations for the areas on which the radiologist commented. The radiologist may review the radiology report, confirm periventricular plaques, add a differential diagnosis (e.g., suggesting follow-up imaging), etc. Thus, the platform 130 reduces report writing time, and provides a structured and detailed radiology report that is ready for approval.

According to another example use case, a radiologist evaluates a CT image for a suspected pulmonary embolism. The platform 130 may receive the CT image, segment the lung with the segmentation model 310, and classify emboli within pulmonary arteries using the classification model 320. The platform 130 may transcribe the radiologist's speech data regarding emboli using the NLP model 330, and generate a radiology report using the AI model 340. The radiology report may additionally mark the appearance of mediastinum and pleura as normal. The radiology report m include detailed descriptions of emboli location, size, and associated findings like pleural effusion. The radiologist may review the radiology report, and make minor adjustments and/or approve the radiology report, which improves the efficiency and time of radiology report generation.

According to another example use case, a radiologist may interpret a challenging abdominal MRI image for suspected hepatocellular carcinoma (HCC). The platform 130 may receive the MRI image, and pre-process the image such as by optimizing for contrast and reducing contrast. The platform 130 may segment the MRI image of the liver with the segmentation model 310, and classify structures (e.g., detected lesions) using the classification model 320. The platform 130 may receive speech data of the radiologist relating to the radiologist's findings, such as speech data identifying lesion size, location, vascular involvement, etc. The platform 130 may generate text data correspond to the speech data using the NLP model 330, and generate a radiology report using the AI model 340 that includes descriptions of normal structures, such as the gallbladder, pancreas, and kidneys. The platform 130 may display an indicator suspecting HCC, and include the radiologist's differential diagnosis, which suggests follow-up imaging and a biopsy. The radiologist approves the radiology report with minimal edit, which may improve comprehensive documentation that is consistent with clinical standards.

According to another example use case, a radiologist assesses a PET-CT image for metastatic breast cancer. The platform 130 may receive the PET-CT image, segment structures (e.g., bones, liver, lungs, etc.) using the segmentation model 310, and classify metastatic lesions using the classification model 320. The platform 130 may receive speech data of the radiologist based on the radiologist verbally commenting on specific metastases in the spine and liver. The platform 130 may generate text data corresponding to the speech data using the NLP model 330. The platform 130 may generate annotations covering other organs as having no abnormal metabolic activity, and generate annotations using the text data. The platform 130 may generate a radiology report using the AI model 340, which includes a summary of metastatic disease extent, standardized uptake values (SUVs), and a recommended follow-up. The radiologist reviews and approves the radiology report, which reduces the time in documenting widespread disease.

According to another example use case, a busy radiology department processes a high volume of mammograms for routine screening. The platform 130 may segment breast tissue using the segmentation model 310, and classify suspicious calcifications and masses using the classification model 320. The platform 130 may receive speech data of the radiologist based on the radiologist verbally commenting on observations on abnormalities. The platform 130 may generate a radiology report using the AI model 340, which includes descriptions of normal breast tissue, axillary lymph nodes, and benign findings. The radiologist may review, adjust, and/or approve the radiology report, which enables rapid throughput, timely patient notification, reduces backlog, and enhances the radiologist department's capacity to handle more screenings.

According to another example use case, a radiologist may interpret follow-up MRI images for previously treated brain tumors. The platform 130 may segment the brain MRI images into relevant structures using the segmentation model 310, and classify residual or recurrent tumor tissue using the classification model 320. The platform 130 may receive speech data of the radiologist based on the radiologist verbally commenting on changes in tumor size, enhancement patterns, and surrounding edema. The platform 130 may generate a radiology report using the AI model 340 which includes sections on stable findings and compares current images with previous images. The radiologist finalizes the radiology reports quickly focusing on critical updates, which enhances reporting efficiency and consistency across multiple follow-up studies.

According to another example use case, a radiology team aims to improve diagnostic accuracy by adapting to the latest research. The platform 130 may integrate a feedback loop, which allows radiologists to rate the accuracy and relevancy of the generated radiology reports. Each reviewed and corrected radiology report is fed back into the platform 130, which updates the segmentation model 310, the classification model 320, the NLP model 330, and/or the AI model 340. This continuous learning approach allows the platform 130 to evolve with new clinical data, thereby improving the ability to detect subtle abnormalities and generate precise radiology reports. Over time, the platform 130 adapts to evolving institutional standards and radiologist's preferences, which permits continuous improvement and learning within the radiology department. The radiology residents may benefit by receiving up-to-date educational content that aligns with current clinical practices.

Conventional approaches may involve manual interpretation. For instance, radiology images, such as MRI images, CT images, PET images, X-ray images, etc., are more often than not manually interpreted by radiologists. This is time-consuming while also being prone to human errors. Radiology report are often manually written descriptive documentation of findings pertaining to a particular radiologists own style, way of writing, and documenting. On a larger scale, this leads to inconsistent report quality and delays. Basic speech recognition may transcribe a radiologist′ comments into unstructured and unstandardized notes, while also lacking information obtained via integration of AI image analysis. Manual interpretation done by radiologists having little to no decision support is a big factor for delays in diagnosis, which impacts patient care and treatment timelines. Manual report writing leads to inconsistencies. This has a significant impact on clarity and accuracy of radiology reports. Basic speech recognition in addition to having no integration with image analysis results in fragmented workflows, which reduces efficiency and increases cognitive load on radiologists.

In contrast, the embodiments herein integrate a segmentation model 310, a classification model 320, an NLP model 330, and an AI model 340. The segmentation model 310 segments medical images and identifies various structures of interest. The classification model 320 detects the structures, and provides a primary analysis of the structures. The platform 130 generates text data corresponding to speech data of a radiologist using the NLP model 330, and generates a radiology report using the AI model 340, which includes the findings from the image analysis and annotations of the radiologist in a structured and detailed documentation format.

The embodiments herein enhance diagnostic accuracy and efficiency by integration of a radiologist's observations and advanced autonomous image analysis, through segmentation & classification, of anomalous areas in generating a radiology report. This significantly enhances accuracy, reduces interpretation time, and improves workflow efficiency.

The embodiments herein provide consistent and structures radiology reports. The embodiments integrate anomalous findings of the radiologist and the segmentation model 310 and the classification model 320, which generates a structured radiology report by auto-filling other areas as being normal while also highlighting critical areas. This ensures consistency and clarity while ensuring that the generated reports conform to a pre-defined documentation standard.

The embodiments herein reduce the workload of radiologists. The embodiments herein integrate a radiologist's verbal comments using the NLP model 330 with autonomous analysis via the segmentation model 310 and the classification model 320. In this way, the embodiments generate accurate and consistent reports that may require minimal changes if any, which greatly reduces cognitive load on radiologists and enhances overall efficiency.

The embodiments herein provide informed decision-making. The AI model 340 provides contextual insights supporting informed decision-making. The integration of the segmentation model 310 and the classification model 320 mitigates potentially missed areas of anomalies in scans. The integration of the segmentation model 310 and the classification model 320 with the NLP model 330 which generates text data corresponding to the radiologist's verbal comments further enhances the robustness of the generated radiology report.

The embodiments herein provide continuous learning and improvement. The embodiments incorporate clinician feedback mechanisms to learn and improve over time, which adapts to evolving clinical needs and optimizes diagnostic processes and outcomes.

The embodiments herein, unlike manual interpretation in conventional systems, integrates advanced AI technologies, such as a segmentation model 310, a classification model 320, an NLP model 330, and an AI model 340, which automates image segmentation and classification, and radiology report generation. The segmentation model 310 segments medical images, and the classification model 320 classifies structures. This autonomous methodology of detecting anomalies enhances accuracy and efficiency, and reduces the time spent on manual interpretation. The AI model 340 generates detailed and comprehensive radiology reports, and integrates the segmentation results and classification results with a radiologist's verbal comments, which produces structured and consistent documentation that surpasses manual reporting. By generating text data corresponding to speech data using the NLP model 330, the system integrates the text data with a segmentation result and a classification result which streamlines radiology report generation, and also reduces cognitive load on radiologists. The system provides real-time diagnostic insights, leverages patient data, and medical literature on which the AI model 340 is trained on, which enables informed decision-making. By incorporating radiologist feedback, the system learns and improves over-time, and adapts to evolving clinical needs, which optimizes diagnostic processes and outcomes. In addition to the radiologist's diagnosis on the medical image, the system also generates a diagnosis based on a segmentation result and a classification result, thereby potentially providing differential diagnosis, which enhances diagnostic accuracy as well as efficiency. The system combines unstructured observations from the radiologist with radiology scans, knowledge determined from research articles, and prior similar cases to generate a complete and holistic diagnostic picture, which thereby increases the robustness of the generated radiology report. The system's real-time diagnostic assistance with patient-specific recommendations reduces workload of radiologists, improves the diagnostic process, reduces the amount of time to provide treatment decisions, and minimizes delays and interruptions in patient care. The system enables medical devices to stay compliant with Health Insurance Portability and Accountability Act (HIPAA) standards, which secures patient data while adhering to best practice frameworks like Center for Internet Security (CIS) security benchmarks. The system may include visualization dashboards for a centralized view of the enterprise level users, and permissions to identify any deviations and remediation to prevent protected health information (PHI) data breach.

The embodiments of the present disclosure may provide AI-driven automation, advanced segmentation and classification, comprehensive radiology report generation, real-time diagnostic insights, real-time diagnosis and treatment, and continuous learning and adaptation. In this way, the embodiments herein provide an improvement in the technical field of radiology, and an improvement in the generation of radiology reports. The embodiments reduces cognitive load reduction on radiologists, reduces workloads and reduces time spent per diagnosis, which thereby improves patient outcomes. The embodiments provide more accurate radiology reports by the usage of the various models in addition to the radiologist's verbal comments, which reduces the likelihood of inaccurate or incomplete radiology reports. By combining different knowledge, the embodiments provide different diagnoses, thereby improving accuracy. The embodiments provide faster and less inexpensive generation of radiology reports by autonomous generation of radiology reports, which greatly reduces time spent by radiologists on report generation.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. A system comprising:
a memory configured to store instructions; and
one or more processors configured to execute the instructions to:
receive a medical image of a region of interest of a subject;
segment a structure of the region of interest of the subject using a segmentation model;
classify the structure of the region of interest of the subject using a classification model;
display the medical image including the classified structure via a user device of a radiologist;
receive speech data, of the radiologist, related to the classified structure from the user device;
generate text data corresponding to the speech data using a natural language processing model;
generate, using an artificial intelligence (AI) model, a radiology report corresponding to the medical image in a predetermined format, wherein the AI model is trained to receive the classified structure and the text data corresponding to the speech data, of the radiologist, related to the classified structure and generate the radiology report in the predetermined format;
identify, using the AI model, the classified structure based on a classification result of the classification model;
identify, using the AI model, the generated text data corresponding to the speech data, of the radiologist, related to the classified structure;
generate, using the AI model, an annotation for the classified structure using the text data based on identifying the generated text data corresponding to the speech data, of the radiologist, related to the classified structure; and
display the radiology report including the medical image and the generated annotation including the generated text data in relation to the classified structure via the user device of the radiologist.

2. The system of claim 1, wherein the one or more processors are further configured to:
identify, using the AI model, that no text data exists for another structure in the medical image; and
generate, using the AI model, another annotation corresponding to the another structure based on predetermined information and based on identifying that no text data exists for the another structure in the medical image.

3. The system of claim 1, wherein the one or more processors are further configured to:
identify, using the AI model, a standardized sentence structure of the predetermined format;
change, using the AI model, a sentence structure of the speech data into the standardized sentence structure to match standardized sentence structure of the predetermined format,
wherein the annotation includes the standardized sentence structure.

4. The system of claim 1, wherein the one or more processors are further configured to:
receive a template that identifies one or more sections of the radiology report,
wherein the generating the radiology report comprises generating the radiology report using the template.

5. The system of claim 1, wherein the medical image in the radiology report includes a segmentation result generated by the segmentation model, and a classification result generated by the classification model.

6. The system of claim 1, wherein the segmentation model is a convolutional neural network that is configured for image segmentation.

7. The system of claim 1, wherein the classification model is a residual neural network.

8. A method comprising:
receiving a medical image of a region of interest of a subject;
segmenting a structure of the region of interest of the subject using a segmentation model;
classifying the structure of the region of interest of the subject using a classification model;
displaying the medical image including the classified structure via a user device of a radiologist;
receiving speech data, of the radiologist, related to the classified structure from the user device;
generating text data corresponding to the speech data using a natural language processing model;
generating, using an artificial intelligence (AI) model, a radiology report corresponding to the medical image in a predetermined format using an artificial intelligence model, wherein the AI model is trained to receive the classified structure and the text data corresponding to the speech data, of the radiologist, related to the classified structure and generate the radiology report in the predetermined format;
identifying, using the AI model, the classified structure based on a classification result of the classification model;
identifying, using the AI model, the generated text data corresponding to the speech data, of the radiologist, related to the classified structure;
generating, using the AI model, an annotation for the classified structure using the text data based on identifying the generated text data corresponding to the speech data, of the radiologist, related to the classified structure; and
displaying the radiology report including the medical image and the generated annotation including the generated text data in relation to the classified structure via the user device of the radiologist.

9. The method of claim 8, further comprising:
identifying, using the AI model, that no text data exists for another structure in the medical image; and
generating, using the AI model, another annotation corresponding to the another structure based on predetermined information and based on identifying that no text data exists for the another structure in the medical image.

10. The method of claim 8, further comprising:
identifying, using the AI model, a standardized sentence structure of the predetermined format;
changing, using the AI model, a sentence structure of the speech data into the standardized sentence structure to match standardized sentence structure of the predetermined format,
wherein the annotation includes the standardized sentence structure.

11. The method of claim 8, further comprising:
receiving a template that identifies one or more sections of the radiology report,
wherein the generating the radiology report comprises generating the radiology report using the template.

12. The method of claim 8, wherein the medical image in the radiology report includes a segmentation result generated by the segmentation model, and a classification result generated by the classification model.

13. The method of claim 8, wherein the segmentation model is a convolutional neural network that is configured for image segmentation.

14. The method of claim 8, wherein the classification model is a residual neural network.

15. A non-transitory computer-readable medium storing instructions that, when executed by one or more processors, cause the one or more processors to:
receive a medical image of a region of interest of a subject;
segment a structure of the region of interest of the subject using a segmentation model;
classify the structure of the region of interest of the subject using a classification model;
display the medical image including the classified structure via a user device of a radiologist;
receive speech data, of the radiologist, related to the classified structure from the user device;
generate text data corresponding to the speech data using a natural language processing model;
generate, using an artificial intelligence (AI) model, a radiology report corresponding to the medical image in a predetermined format, wherein the AI model is trained to receive the classified structure and the text data corresponding to the speech data, of the radiologist, related to the classified structure and generate the radiology report in the predetermined format;
identify, using the AI model, the classified structure based on a classification result of the classification model;
identify, using the AI model, the generated text data corresponding to the speech data, of the radiologist, related to the classified structure;
generate, using the AI model, an annotation for the classified structure using the text data based on identifying the generated text data corresponding to the speech data, of the radiologist. related to the classified structure; and
display the radiology report including the medical image and the generated annotation including the generated text data in relation to the classified structure via the user device of the radiologist.

16. The non-transitory computer-readable medium of claim 15, wherein the one or more processors are further configured to:
identify, using the AI model, that no text data exists for another structure in the medical image; and
generate, using the AI model, another annotation corresponding to the another structure based on predetermined information and based on identifying that no text data exists for the another structure in the medical image.

17. The non-transitory computer-readable medium of claim 15, wherein the one or more processors are further configured to:
identify, using the AI model, a standardized sentence structure of the predetermined format;
change, using the AI model, a sentence structure of the speech data into the standardized sentence structure to match standardized sentence structure of the predetermined format,
wherein the annotation includes the standardized sentence structure.

18. The non-transitory computer-readable medium of claim 15, wherein the one or more processors are further configured to:
receive a template that identifies one or more sections of the radiology report,
wherein the generating the radiology report comprises generating the radiology report using the template.

19. The non-transitory computer-readable medium of claim 15, wherein the segmentation model is a convolutional neural network that is configured for image segmentation.

20. The non-transitory computer-readable medium of claim 15, wherein the classification model is a residual neural network.
